(19) United States
(12) Patent Application Publication
(10) Pub. No.: US 2021/0257061 A1
(43) Pub. Date: Aug. 19, 2021

(54) METHOD AND SYSTEM FOR DEVELOPING CLINICAL TRIAL PROTOCOLS

(71) Applicant: Gen LI, East Lyme, CT (US)

(72) Inventor: Gen LI, East Lyme, CT (US)

(21) Appl. No.: 17/265,414

(22) PCT Filed: Aug. 8, 2019
(86) PCT No.: PCT/US2019/045789

§ 371 Date: Feb. 2, 2021

Related U.S. Application Data
(60) Provisional application No. 62/716,019, filed on Aug. 8, 2018.

(51) Int. Cl. G16H 10/20; G16H 15/00; G16H 50/20; G16H 70/60

(52) U.S. Cl. CPC G16H 10/20; G16H 70/60; G16H 50/20; G16H 15/00

(57) ABSTRACT

The present invention discloses methods and systems for developing clinical trial protocols, in particular, the inclusion/exclusion criteria used to define targeted patient population. In some embodiments, the present invention provides methods and systems to develop and/or optimize the inclusion/exclusion criteria based on quantitative analysis. In some embodiments, the methods and systems of the present invention allow to achieve the objectives of a clinical trial.

### CROSS-REFERENCE TO RELATED APPLICATIONS

This application claims the benefit of U.S. Provisional Application No. 62/716,019, filed Aug. 8, 2018. The entire contents and disclosures of the preceding application are incorporated by reference into this application.

Throughout this application, various publications are cited. The disclosures of these publications in their entireties are hereby incorporated by reference into this application to more fully describe the state of the art to which this invention pertains.

### FIELD OF THE INVENTION

The present invention relates to clinical trial protocol development, in particular, inclusion/exclusion criteria.

### BACKGROUND OF THE INVENTION

Clinical trials are the workhorses of the pharmaceutical industry. They are the basis of safe and effective use for new therapies. Clinical trials are the final stage of pharmaceutical development and a lot depends on the quality and interpretability of their results. Surprisingly, despite thousands of clinical trials being performed every year, they often take longer than expected with poor patient enrollment being a common reason for stopping trials early. The reason that a clinical trial runs into trouble is usually simple: the investigator sites are not enrolling patients as fast as planned or cannot find patients to enroll at all. The root causes for patient enrollment difficulties are much more complicated and challenging to tease out. Therefore, it is highly desirable to have an innovative platform to assess multiple variables impacting patient enrollment in an integrated fashion. These variables usually fall into one of the following major categories:

Whether the enrollment of subjects is being or likely being channeled to a competing trial;

Development of a protocol and assessment of whether the protocol is feasible;

Whether the investigator sites have performed on par with other similar trials;

Execution of the site activation plan;

With answers for the above questions, whether the planned enrollment curve is realistic.

The present invention provides a technical solution for the development and/or assessment of a feasible protocol based on a targeted patient population.

Each clinical trial is guided by a protocol. Determination of inclusion/exclusion criteria is an important component of a protocol design. In general, inclusion/exclusion criteria include such criteria as age, gender, disease indication specifics etc. The inclusion/exclusion criteria help users to define the patient population. For example, diabetes protocols usually include relevant biochemical parameters such as Hemoglobin A1c concentration in blood. In current clinical development practice, as shown in FIG. 1, the determination of a set of protocol patient inclusion/exclusion criteria highly depends on the experience of the medical professional(s) responsible for the development of the protocol and on institutional learning of the clinical development organization sponsoring the clinical trials. Conventionally, a long period (e.g., 6-12 months) is needed to develop a protocol, which is often inconclusive and inconsistent, resulting in multiple rounds of protocol amendments. It means that the design of the protocol must be modified during the process of execution. This is financially costly and significantly delays the time for a clinical trial to reach a final conclusion (either approval or rejection to a set of statistical assumptions). In addition, the determination of inclusion/exclusion criteria based on experience of multiple people (or other sources) with different backgrounds or training may make the final product, i.e., the protocol, far away from the objectives of the clinical trial. It may even lead to the failure of the entire clinical trial. Furthermore, there is no quantitative way to standardize the inputs from different sources such as references, expert opinions and objectives of the clinical trial. Thus, there is a critical need for an innovative platform to consistently and reliably assess multiple variables in an integrated fashion for the development of a clinical trial protocol.

### SUMMARY OF THE INVENTION

The present invention provides methods and systems for developing clinical trial protocols, in particular, the inclusion/exclusion criteria used to define targeted patient population.

In one embodiment, the present invention hereby provides a method and a system to develop and/or optimize the inclusion/exclusion criteria based on quantitative analysis.

In one embodiment, the present invention discloses a system for developing a set of inclusion/exclusion criteria for a target clinical trial related to a disease or a condition, the system comprising:
a storage unit;
a computing unit;
an output unit; and
an input unit, all operable together,
wherein a filter comprising a set of filtering parameters is provided through the input unit;
wherein the filter is applied to a master database comprising historical data on clinical trials to create a sub-database in the storage unit, the sub-database comprising historical data on clinical trials related to the disease or the condition and having sufficient data for subsequent analysis,
wherein the computing unit conducts the steps of:
   a) selecting parameters in the sub-database to obtain a number of selected parameters, and
   b) conducting an analysis on the selected parameters to determine their desirable values, wherein the analysis comprises:
      1) a frequency analysis to determine the frequency with which a value associated with a selected parameter has been used in the clinical trials in the sub-database, and
      2) a quantitative analysis to quantify a risk associated with selecting a value associated with a selected parameter, or a risk associated with selecting multiple values, wherein each of the multiple values is associated with one of the selected parameters; wherein a value associated with a selected parameter and an acceptable risk is a desirable value, and wherein one or more selected parameters with their desirable values define a set of inclusion/exclusion criteria; and
wherein the output unit transmits and displays the set of inclusion/exclusion criteria.

In one embodiment, sufficient number of clinical trials and patients required for subsequent analysis means that there are sufficient data to conduct analysis(es) to reach a result with a statistical meaning. In one embodiment, the sufficient number required for subsequent analysis depends on other factors, such as the disease or condition under investigation, the historical data of clinical trials, and the objectives of the target clinical trial. However, the interpretation of “sufficient”, “sufficiency” and other equivalents shall include without limitation the ranges as typically shown in the examples of the present invention.

In one embodiment, the present invention discloses a method of developing a set of inclusion/exclusion criteria for a clinical trial related to a disease or a condition, the method comprising:
a) applying a filter to a master database containing historical data on clinical trials to create a sub-database, wherein the filter comprises a set of filtering parameters, and the sub-database contains clinical trials related to the disease or condition, and has sufficient data for subsequent analysis,
b) selecting parameters that fit the objectives of the target clinical trial from the clinical trials in the sub-database to obtain a number of selected parameters;
c) conducting an analysis to determine desirable values of the selected parameters, wherein the analysis comprises:
   1) a frequency analysis to identify the frequency with which a value associated with a selected parameter has been used in the clinical trials in the sub-database, and
   2) a quantitative analysis to quantify a risk associated with selecting a value for such selected parameter, or a risk associated with selecting values for such selected parameters, wherein each of the multiple values is associated with one of the selected parameters; wherein a value associated with a selected parameter and an acceptable risk is a desirable value, and wherein multiple selected parameters with their desirable values define a set of inclusion/exclusion criteria, and
d) outputting the set of inclusion/exclusion criteria.

In summary, the present invention provides methods and systems to develop or design a feasible clinical trial protocol by quantitatively analyzing historical data. In one embodiment, the present invention provides a method and a system to identify the values for a set of selected parameters to be used as inclusion/exclusion criteria. In one embodiment, the present invention provides a method and a system to develop and/or optimize the inclusion/exclusion criteria based on quantitative analysis. In one embodiment, the present invention discloses a method and a system to align the objective of a clinical trial with the quantitative analysis of potential risks. In one embodiment, the present invention discloses a method and a system that can quickly develop final inclusion/exclusion criteria for a reliable high-quality clinical protocol with consistency, objectivity, verifiability and within a shorter period of time. In one embodiment, the method and the system can establish final inclusion/exclusion criteria for a clinical protocol within a period of less than 2 months. In one embodiment, the method and the system can establish final inclusion/exclusion criteria for a clinical protocol within a period of less than 1 month. In one embodiment, a disease or a condition is a metabolic disease or condition, a respiratory disease condition, or a neurologic disease condition, and other diseases or conditions studied by randomized clinical trials.

### BRIEF DESCRIPTION OF THE FIGURES

FIG. 1 is a diagram schematically demonstrating a process typically used in the field for designing a clinical trial.

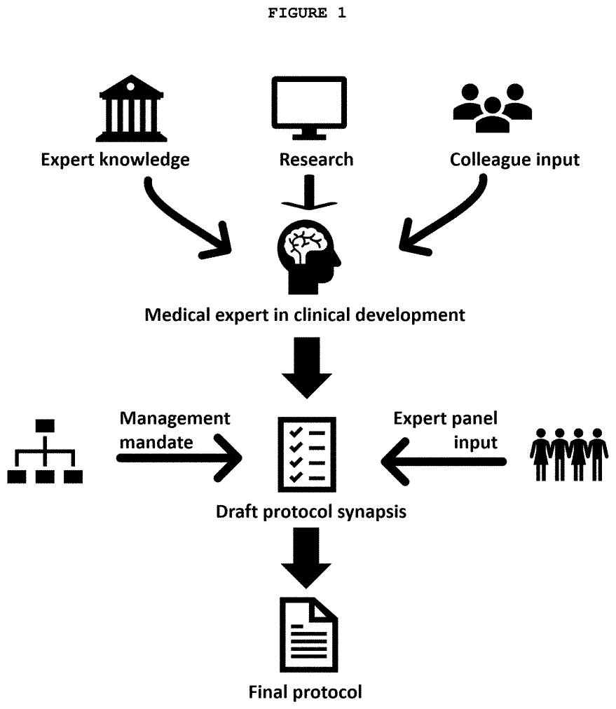

FIG. 2 shows the creation of a sub-database according to one embodiment of the present invention.

FIGS. 3A and 3B show the selection of parameters and determination of the mode values and desirable values, respectively, according to one embodiment of the present invention.

FIGS. 4A and 4B show typical calculations of distance according to one embodiment of the present invention.

FIG. 5 shows the distribution of patients at baseline by Eastern Cooperative Oncology Group (ECOG) score according to one embodiment.

FIG. 6A is a bubble chart showing the relationship between Gross Site Enrollment Rate (GSER) and the number of Investigator Sites (N) (the bubble/circle size indicates the enrollment cycle time (ECT) in the clinical trial) for Phase II NSCLC clinical trials. FIG. 6B shows a formula quantitatively describing the relationship between GSER and N for Phase II NSCLC clinical trials.

FIG. 7A is a GSER bubble chart showing the relationship between GSER and N (the bubble/circle size indicates enrollment cycle time (ECT) in the clinical trial) for the same set of Phase II NSCLC clinical trials. FIG. 7B shows a formula quantitatively describing the relationship between GSER and N.

### DETAILED DESCRIPTION OF THE INVENTION

The present invention provides methods and systems for developing clinical trial protocols, in particular, the inclusion/exclusion criteria used to define targeted patient population.

In one embodiment, the present invention hereby provides a method and a system to develop and/or optimize the inclusion/exclusion criteria based on quantitative analysis. In one embodiment, the present invention allows to align the objectives of the clinical trial with the quantitative analysis of potential risks. In one embodiment, one of the objectives of the clinical trial is to complete patient enrollment within a short period with little consideration given to other factors such as Gross Site Enrollment Rate (GSER) and Site Effectiveness Index (SEI). In one embodiment, some of the objectives of the clinical trial are to ensure a relatively high level of GSER and SEI so as to keep the budget within a range. In one embodiment, the objective of the target clinical trial is to balance multiple factors by assigning them with different weights.

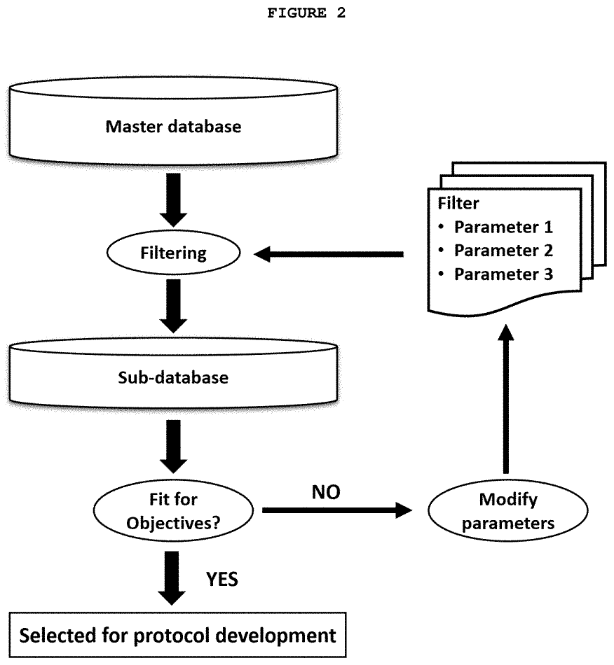

In one embodiment, in order to create a sub-database, a filter containing pre-set parameters fitting objectives and features of a clinical trial for which the protocol is being developed is applied to a master database. In one embodiment, as represented in FIG. 2, the filter is subject to further adjustment until a sub-database fully representing the objectives is obtained. In one embodiment, there is a possibility that no sub-database can be obtained, which indicates that achieving the objectives as originally planned may face some challenges or high risks and some of the objectives may need further adjustment. In one embodiment, the sub-database comprises sufficient data and information for statistical analysis. In one embodiment, a sub-database contains sufficient number of clinical trials to provide statistically meaningful analysis results. In one embodiment, when the sub-database contains a massive volume of data, much more than what is deemed necessary, the subsequent analysis is performed with the most relevant and/or recent data/information with appropriate volume.

In one embodiment, the parameters included in the filter used for creating the sub-database include, but are not limited to, type/stage of disease/disorder, age and gender of patients, phase of the clinical trial, country, number of patients, number of investigator sites, Enrollment Cycle Time (ECT), Site Effectiveness Index (SEI), Adjusted Site Enrollment Rate (ASER). In one embodiment, the filter is pre-set by user. In one embodiment, one or more parameters of the filter are further modified in view of the objectives of the target clinical trial. In one embodiment, one or more parameters of the filter are further modified so as to obtain sufficient data for subsequent analysis (analyses).

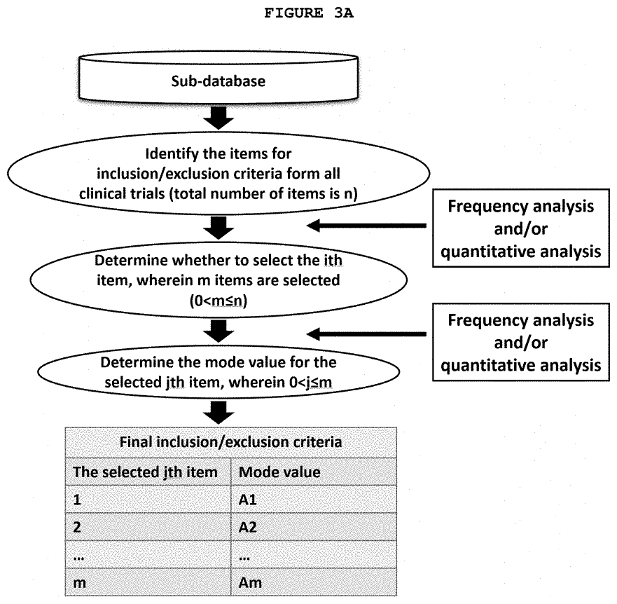

In one embodiment, inclusion/exclusion criteria are generated from a sub-database according to FIG. 3A or FIG. 3B. First, a frequency or quantitative analysis determines whether a parameter should be included. Second, the value is determined by a subsequent frequency and/or quantitative analysis. In one embodiment, the desirable value (selected value) equals to the mode value. In one embodiment, a parameter that has been used in at least 50% of clinical trials in the sub-database is selected for such inclusion/exclusion criteria. In one embodiment, if a parameter fits the objectives of the target clinical trial, i.e., the risk associated with such parameter selection is acceptable, it may be selected though it has been used in less 50% of clinical trials. In one embodiment, the acceptable risk refers to the level of risk as quantified by the quantitative analysis that is within the desired level or range in view of the objectives of the target clinical trial. In one embodiment, the objectives of the target clinical trial may have different priorities, e.g., a sponsor may put the time of completing patient enrollment as the highest priority and not be sensitive to the overall cost.

In one embodiment, the quantitative analysis is conducted by comparing the operational outcomes (characters) of clinical trials in the sub-database to operational outcomes of clinical trials at baseline. In one embodiment, there are sufficient data so that a relationship with a statistical meaning can be established. In one embodiment, the quantifiable operational outcomes include, without limitation, one or more of the following: number of patients, number of investigator sites (N), enrollment cycle time (ECT), Gross Site Enrollment Rate (GSER), Site Effectiveness Index (SEI), Adjusted Site Enrollment Rate (ASER).

As used herein, Site Effectiveness Index (SEI) is defined as:

$$S E I = \frac{{\int }_{i = 1}^{N} ( {Et}_{i} - {St}_{i} )}{( {Et}_{s} - {St}_{s} ) \times {N}_{max}} ,$$

wherein Etᵢ is the time (date) site i closed for patient enrollment, Stᵢ is the time (date) site i opened for patient enrollment, Nₘₐₓ is the maximum number of investigator sites opened for enrollment during the patient enrollment of the study (trial), Etₛ is the time (date) clinical study (trial) closed for patient enrollment, Stₛ is the time (date) clinical study (trial) opened for patient enrollment. Etₛ is the time (date) clinical study (trial) ended for patient enrollment.

In one embodiment, a mathematical expression of Enrollment Cycle Time (ECT), i.e. the period starting from enrollment opening date and ended with enrollment closing date, (Etₛ−Stₛ) is:

Enrollment Cycle Time=Total Enrollment/[(Gross Site Enrollment Rate(GSER)×(Maximum Number of Investigator Sites(Nₘₐₓ)],

wherein the GSER is related to site selection (performance), among other things, and SEI is related to study startup (process).

In one embodiment, the relationship between Site Effectiveness Index (SEI) and other variables, such as Enrollment Cycle Time (ECT), can be described as:

ECT=TE/[ASER×SEI×Nₘₐₓ],

wherein the Adjusted Site Enrollment Rate (ASER) is defined as:

$$A S E R = \frac{T E}{{\int }_{i = 1}^{N} ( {Et}_{i} - {St}_{i} )}$$

wherein TE is Total Enrollment. When the target clinical trial is in the planning stage, TE refers to the targeted total number of patients to be enrolled in the target clinical trial. For an evaluation of historical data, TE is the total number of patients actually enrolled in a clinical trial.

### Parameter Selection

In one embodiment, the present invention discloses a method and a system for clinical trial protocol development. Clinical trial protocols may include different parameters. For example, a lower age limit may be included as a parameter for a protocol template for certain clinical trials. In one embodiment, the present invention discloses a method and a system to identify the parameters to be selected as inclusion/exclusion criteria. In one embodiment, the frequency with which a parameter has been used in clinical trials is calculated according to equation (1):

$$F = \frac{{N}_{w}}{{N}_{w} + {N}_{wo}} , \qquad ( 1 )$$

wherein N_{w} is the number of clinical trials with such parameter and N_{wo} is the number of clinical trials without such parameter.

In one embodiment, the frequency is calculated by considering the weight of the enrolled patients number according to equation (2):

$$F = \frac{{N}_{w} * {f}_{w}}{{N}_{w} * {f}_{w} + {N}_{wo} {f}_{wo}} , \\ wherein {f}_{w} = \frac{\begin{matrix}Number of patients enrolled \\ in clinical trials with the item\end{matrix} }{Total number of patients in clinical trials with such item} , \\ {f}_{wo} = \frac{\begin{matrix}Number of patients enrolled \\ in clinical trial without the item\end{matrix} }{Total number of patients in clinical trials with such item} , and {f}_{w} + {f}_{wo} = 1.0 . \qquad ( 2 )$$

In one embodiment, when F is equal to or larger than 0.5 or 50%, the parameter is selected as one inclusion/exclusion parameter (selected parameter) for the protocol development. In one embodiment, a parameter can be removed when a quantitative analysis indicates that no or very limited difference is observed when comparing with the results without such parameter. In one embodiment, a parameter can be kept or added when a quantitative analysis indicates that the results with such parameter fits the objectives of the clinical trial, even if such parameter has been used in less than 50% of clinical trials historically. In one embodiment, the significant benefits due to such parameter selection include, but are not limited to, shorter ECT, higher enrollment rate, more clearly defined population.

In one embodiment, the present invention disclosed a method to rank the values according to the frequency (F).

### Value Determination for a Selected Parameter

In one embodiment, the value for a selected parameter is determined according to a frequency analysis. Assuming the parameter value (x) can be selected from a group of values aᵢ, wherein i is an integer ranging from 1 to p, the frequency can be calculated according to equation (3):

$${F}_{ai} = \frac{{N}_{x = ai}}{Total number of clinical trials with such item} , \qquad ( 3 )$$

wherein Nₓ₌ₐᵢ is the number of clinical trials in which the parameter value (x) is aᵢ.

In one embodiment, the frequency analysis is a weight-average frequency and can be calculated according to equation (4):

$${F}_{ai} = \frac{{N}_{x = ai} * {f}_{x = ai}}{{\sum }_{i = 1}^{p} {N}_{x = ai} * {f}_{x = ai}} , \\ wherein {f}_{x = ai} = \frac{\begin{matrix}Number of patients enrolled in \\ clinical trials with an item value of ai\end{matrix}}{\begin{matrix}Total number of patients \\ in clinical trials with such item\end{matrix} } , and \\ {\sum }_{i = 1}^{p} {f}_{x = ai} = 1.0 . \qquad ( 4 )$$

In one embodiment, the percentage of patients enrolled in clinical trials with a parameter value of a1 is calculated according to

$${f}_{x = a 1} = \frac{\begin{matrix}Number of patients enrolled in \\ clinical trials with an item value of a 1\end{matrix} }{Total number of patients in clinical trials with such} ,$$

In one embodiment, when the frequency of a value for a selected parameter is the largest, the value is selected as the desirable value for the selected parameter. In one embodiment, the desirable value is equal to the mode value. In one embodiment, the value can be further adjusted when a quantitative analysis indicates that such adjustment fits the objectives or certain objectives with high priority of the target clinical trial. In one embodiment, such adjustment may result in, for example, shorter ECT, higher GSER, more clearly defined population.

### Quantitative Analysis

In some embodiments, the risk of selecting a value for a selected parameter or a set of inclusion/exclusion criteria for the target clinical trial can be assessed or calculated. In some embodiments, the risk means an impact of selecting a value for a selected parameter or a set of inclusion/exclusion criteria on achieving an objective of a clinical trial based on the analysis of historical data.

In one embodiment, the risk corresponding to choosing a value compared to choosing some other value, for example, mode value, is quantified by the impact of the choice on one or more operational outcomes (characters) of the objectives of the target clinical trial, wherein the operational outcomes (characters) include but are not limited to GSER, N, ECT, SEI and other quantifiable measurements or outcomes. In one embodiment, the objectives of the target clinical trial also include the enrollment budget and the overall budget for clinical trial, which may be derived from or closely related to these quantifiable measurements.

In one embodiment, the mode value corresponds to the most ideal situation, i.e., a situation with the minimum risk. In one embodiment, the mode value is not necessarily the most ideal situation. In one embodiment, if one objective of the target clinical trial is to complete patient enrollment within a shorter period (a small value of ECT), when the selection of one value rather than another leads to a smaller ECT, it indicates a lower risk; when it leads to a bigger ECT, it indicates a higher risk. In one embodiment, if one objective of the target clinical trial is to complete patient enrollment within a limited budget and reasonable enrollment period (typically a high value of GSER and a small value of N and TE), when the selection of one value rather than another decreases N/TE while the ECT is within the reasonable enrollment period, it indicates a lower risk; otherwise, it indicates a higher risk or uncertainty.

In one embodiment, the risk or uncertainty of a clinical trial protocol, in particular, each of the inclusion/exclusion criteria, can be quantitatively measured. In one embodiment, the graph of Investigator Sites (N) vs Gross Site Enrollment Rate (GSER) can be fitted by the following formulas:

GSER=a*e^{bN}+c, or GSER=a*N^{b}+c,

wherein a, b, and c are constant parameters for a set of clinical trials for a disease or condition; b is a negative constant for a set of clinical trials. In one embodiment, the lower limit of site level enrollment rate is c.

In one embodiment, the GSER is related to Site Effectiveness Index (SEI) and Adjusted Site Enrollment Rate (ASER) as: GSER=SEI×ASER.

In one embodiment, the relationship between GSER and N for the clinical trials can be obtained by a regression analysis using GSER=a*N^{b}+c based on all data in the sub-database, wherein a, b, and c are constant parameters for clinical trials.

In one embodiment, though the relationship between variables (e.g., GSER and N) can be described by different equations, the best fitting equation is selected for the quantitative analysis.

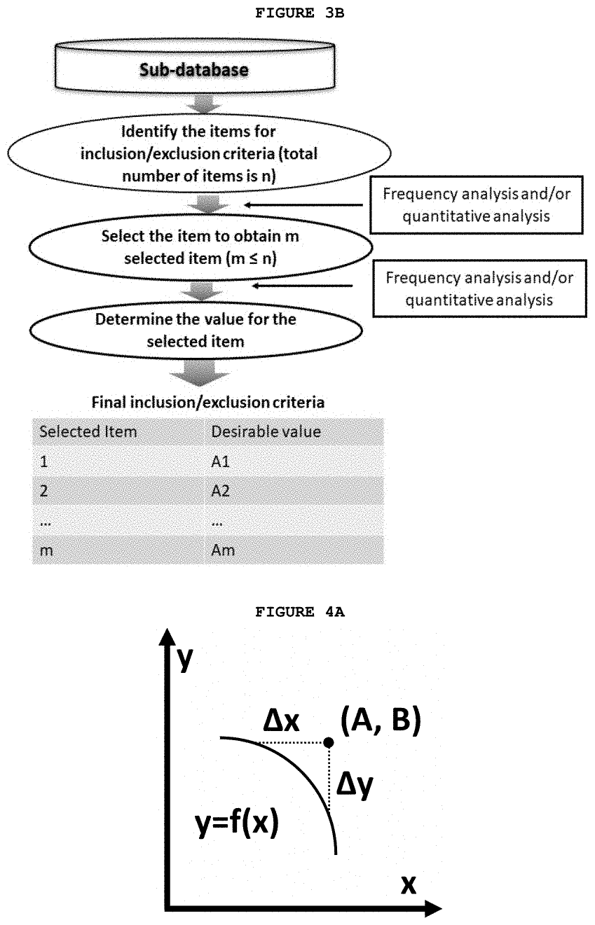

In one embodiment, the risk (K) associated with a point corresponding to a clinical trial with a set of inclusion/exclusion criteria is quantitatively evaluated by calculating the distance to the best fitted equation (the curve). A longer distance from the curve indicates a higher risk. In one embodiment, as shown in FIG. 4A, the distance (D) from a point (P) with coordinates (A, B) to the curve is calculated by

$$D = \frac{\vert Δ x \vert + \vert Δ y \vert}{2} .$$

In one embodiment, the distance (D) from a point (P) with coordinates (A, B) to the curve is calculated by

$$D = \sqrt{\frac{( Δ {x}^{2} + Δ {y}^{2} )}{2}} .$$

In one embodiment, as shown in FIG. 4B, the distance (D) from a point (P) with coordinates (A, B) to the point Q on the curve C, where C=(x(t), y(t)) is calculated by:

$$D ( P , C ) = {min}_{t} \sqrt{{[ A - x ( t ) ]}^{2} + {[ B - y ( t ) ]}^{2}} .$$

In one embodiment, the distance (D) of a point (P) with coordinates (A, B) corresponding to a clinical trial with a set of inclusion/exclusion criteria is the shortest distance from the curve.

In some embodiments, there may be more than one clinical trial with a particular set of inclusion/exclusion criteria. In such embodiments, median or average distance to the curve is calculated for all of clinical trials with a particular set of inclusions/exclusion criteria. In one embodiment, the data of the historical clinical trials meeting the set of inclusion/exclusion criteria are averaged as a single point prior to the calculation of the risk or distance. In some embodiments, there may be no historical clinical trials exactly meeting the particular set of inclusion/exclusion criteria. In such embodiments, data from historical clinical trials partially meeting the particular set of inclusion/exclusion criteria are used to calculate the risk or distance.

In one embodiment, a median distance is calculated by analyzing all points in historical data and can be further used for quantification of risk. In one embodiment, a distance that is longer than the median distance indicates a higher-than-median risk. In one embodiment, a distance that has a statistical significance in comparison to the average indicates a statistically significant risk.

In one embodiment, the interplay among two or more factors is quantitatively evaluated. In one embodiment, the interplay is evaluated by mapping out an overall risk corresponding to each possible set of inclusion/exclusion criteria comprising the selected parameters. In one embodiment, the final set of inclusion/exclusion criteria selected for the target clinical trial is the one with minimum or acceptable risk.

In one embodiment, the present invention discloses a system for developing a set of inclusion/exclusion criteria for a target clinical trial related to a disease or a condition, the system comprising:
a storage unit;
a computing unit;
an output unit; and
an input unit, all operable together,
wherein a filter comprising a set of filtering parameters is provided through the input unit;
wherein the filter is applied to a master database comprising historical data on clinical trials to create a sub-database in the storage unit, the sub-database comprising historical data on clinical trials related to the disease or the condition and having sufficient data for subsequent analysis,
wherein the computing unit conducts the steps of:
   a) selecting parameters in the sub-database to obtain a number of selected parameters, and
   b) conducting an analysis on the selected parameters to determine their desirable values, wherein the analysis comprises:
      1) a frequency analysis to determine the frequency with which a value associated with a selected parameter has been used in the clinical trials in the sub-database, and
      2) a quantitative analysis to quantify a risk associated with selecting a value associated with a selected parameter, or a risk associated with selecting multiple values, wherein each of the multiple values is associated with one of the selected parameters; wherein a value associated with a selected parameter and an acceptable risk is a desirable value, and wherein one or more selected parameters with their desirable values define a set of inclusion/exclusion criteria; and
   wherein the output unit transmits and displays the set of inclusion/exclusion criteria.

In one embodiment, the filter comprises at least one parameter selected from the group consisting of type/stage of disease/disorder, age, gender, phase of the clinical trial, country, number of clinical trials, number of patients, number of investigator sites, enrollment cycle time, Site Effectiveness Index (SEI), Adjusted Site Enrollment Rate (ASER), Gross Site Enrollment Rate (GSER), and any other parameters that can be used to characterize clinical trials.

In one embodiment the the frequency in the frequency analysis is calculated according to

$${F}_{ai} = \frac{{N}_{x = ai}}{Total number of clinical trials with such item} , or$$

$${F}_{ai} = \frac{{N}_{x = ai} * {f}_{x = ai}}{{\sum }_{i = 1}^{p} {N}_{x = ai} * {f}_{x = ai}}$$

wherein Nₓ₌ₐᵢ refers to the number of clinical trials in which the parametric value (x) is aᵢ (i≤p),

$${f}_{x = ai} = \frac{\begin{matrix}Number of patients enrolled \\ in clinical trials with an item value of ai\end{matrix}}{\begin{matrix}Total number of patients \\ in clinical trials with such item\end{matrix} } , and$$

$${\sum }_{i = 1}^{p} {f}_{x = ai} = 1.0 ,$$

wherein p is the total number of values for such parameter in the subdatabase.

In one embodiment, the quantitative analysis analyzes changes in one or more characters that result from trying different values for one or more selected parameters; wherein the one or more characters are selected from the group consisting of number of clinical trials, number of patients, number of investigator sites, enrollment cycle time, Site Effectiveness Index (SEI), Adjusted Site Enrollment Rate (ASER), Gross Site Enrollment Rate (GSER), and any other parameters that can be used to characterize clinical trials.

In one embodiment, the changes in the one or more characters are evaluated by using an equation that quantitatively describes a relationship among variables.

In one embodiment, the equation is selected from the group consisting of:

GSER=a*e\hat{ }bN+c, and

GSER=a*N\hat{ }b+c,

wherein a, b, and c are constants for the clinical trials in the subdatabase and can be determined by a regression analysis of all data in the subdatabase.

In one embodiment, the distance between a point corresponding to a clinical trial with the set of inclusion/exclusion criteria and a curve corresponding to the equation is used to quantitatively describe the risk of the clinical trial.

In one embodiment, the one or more of the desirable values are most frequently used in the clinical trials in the sub-database.

In one embodiment, the equation for a Phase II clinical trial related to non-small cell lung cancer is GSER=2.5394*N^{-0.738.}

In one embodiment, the one or more of the selected parameters in step a) have been used in at least 50% of the clinical trials in the sub-database.

In one embodiment, the present invention discloses a method of developing a set of inclusion/exclusion criteria for a clinical trial related to a disease or a condition, the method comprising:
a) applying a filter to a master database containing historical data on clinical trials to create a sub-database, wherein the filter comprises a set of filtering parameters, and the sub-database contains clinical trials related to the disease or condition, and has sufficient data for subsequent analysis,
b) selecting parameters that fit the objectives of the target clinical trial from the clinical trials in the sub-database to obtain a number of selected parameters;
c) conducting an analysis to determine desirable values of the selected parameters, wherein the analysis comprises:
   1) a frequency analysis to identify the frequency with which a value associated with a selected parameter has been used in the clinical trials in the sub-database, and
   2) a quantitative analysis to quantify a risk associated with selecting a value for such selected parameter, or a risk associated with selecting values for such selected parameters, wherein each of the multiple values is associated with one of the selected parameters; wherein a value associated with a selected parameter and an acceptable risk is a desirable value, and wherein multiple selected parameters with their desirable values define a set of inclusion/exclusion criteria, and
outputting the set of inclusion/exclusion criteria.

In one embodiment, the filter comprises at least one filtering parameter selected from the group consisting of type/stage of disease/disorder, age, gender, phase of the clinical trial, country, number of clinical trials, number of patients, number of investigator sites, enrollment cycle time, Site Effectiveness Index (SEI), Adjusted Site Enrollment Rate (ASER), Gross Site Enrollment Rate (GSER), and any other parameters that can be used to characterize clinical trials.

In one embodiment, the frequency in the frequency analysis is calculated according to

$${F}_{ai} = \frac{{N}_{x = ai}}{Total number of clinical trials with such item} , or$$

$${F}_{ai} = \frac{{N}_{x = ai} * {f}_{x = ai}}{{\sum }_{i = 1}^{p} {N}_{x = ai} * {f}_{x = ai}}$$

wherein Nₓ₌ₐᵢ refers to the number of clinical trials in which the parametric value (x) is aᵢ (i≤p),

$${f}_{x = ai} = \frac{\begin{matrix}Number of patients enrolled \\ in clinical trials with an item value of ai\end{matrix} }{\begin{matrix}Total number of patients \\ in clinical trials with such item\end{matrix} } , and$$

$${\sum }_{i = 1}^{p} {f}_{x = ai} = 1.0 ,$$

wherein p is the total number of values for such parameter in the subdatabase.

In one embodiment, the quantitative analysis is conducted by quantitatively analyzing changes in one or more characters that result from trying different values, wherein the one or more characters are selected from the group consisting of number of clinical trials, number of patients, number of investigator sites, enrollment cycle time, Site Effectiveness Index (SEI), Adjusted Site Enrollment Rate (ASER), Gross Site Enrollment Rate (GSER), and any other parameters that can be used to characterize clinical trials.

In one embodiment, the changes in one or more characters are evaluated by using an equation that quantitatively describes a relationship among variables.

In one embodiment, the equation is selected from the group consisting of:

GSER=a*e^{bN}+c, and GSER=a*N^{b}+c,

wherein a, b, and c are constants for the clinical trials in the sub-database and can be determined by a regression analysis of all data in the sub-database.

In one embodiment, the distance between a point corresponding to a clinical trial with the set of inclusion/exclusion criteria and a curve corresponding to the equation is used to quantitatively describe the risk of the clinical trial.

In one embodiment, one or more of the desirable values are most frequently used in the clinical trials in the sub-database.

In one embodiment, the equation for a Phase II clinical trial related to non-small cell lung cancer is GSER=2.5394*N^{-0.738}.

In one embodiment, one or more of the selected parameter parameters in step b) have been used in at least 50% of the clinical trials in the sub-database.

In one embodiment, the present invention discloses a system for developing a set of inclusion/exclusion criteria for a clinical trial related to a disease or a condition, the system comprising:
a storage unit containing a master database comprising historical data on clinical trials;
a computing unit;
an output unit; and
an input unit, all operable together;
wherein a filter is provided through the input unit;
wherein the computing unit applies the filter to the master database to create a sub-database in the storage unit, the sub-database containing clinical trials related to the disease or condition and having sufficient data for subsequent analysis,
wherein the computing unit conducts the steps of:
   a) selecting parameters in the sub-database to obtain a number of selected parameters fitting the objectives, and
   b) conducting an analysis on the selected parameters to determine their desirable values, wherein the analysis comprises:
      a frequency analysis to determine the frequency with which a value associated with a selected parameter has been used in the clinical trials in the sub-database, wherein a value that is most frequently used in the clinical trials in the sub-database is selected as a desirable value for the selected parameter, and wherein multiple selected parameters with their desirable values define a set of inclusion/exclusion criteria;
wherein the output unit transmits and displays the set of inclusion/exclusion criteria.

In one embodiment, the one or more of the selected parameters are present in at least 50% of the clinical trials in the sub-database.

### EXAMPLES

The invention will be better understood by reference to the Experimental Details which follow, but those skilled in the art will readily appreciate that the specific experiments detailed are only illustrative, and are not meant to limit the invention as described herein, which is defined by the claims which follow thereafter.

Throughout this application, various references or publications are cited. Disclosures of these references or publications in their entireties are hereby incorporated by reference into this application in order to more fully describe the state of the art to which this invention pertains. It is to be noted that the transitional term “comprising”, which is synonymous with “including”, “containing” or “characterized by”, is inclusive or open-ended and does not exclude additional, un-recited elements or method steps.

### Example 1

A sub-database for non-small cell lung cancer (NSCLC) clinical trials is created by filtering a master database containing clinical trials data. The filter contains the following parameters:

a) The disease/disorder is NSCLC;

b) It is a Phase II clinical trial;

c) Each clinical trial has randomized 99 to 201 patients; and

d) Each clinical trial has a total number of investigator sites in a range of 10-96.

A total of 178 clinical trials were selected and included in the sub-database, which was further used to establish the inclusion/exclusion criteria for the protocol.

### Example 2

With the sub-database from Example 1, the frequency for each value of each item may be calculated. The mode value, which is the value with the highest frequency may then be identified. In one embodiment, the desirable value corresponding to the minimum risk is equal to the mode value

Identification of Value for Lower Age Limit: There are 163 trials in the sub-database that include Lower Age Limit as a parameter. Among them, a Lower Age Limit of 18 (i.e., the age of a patient is 18 years or older) was specified in 148 trials. The mode value for Lower Age Limit is “18” as it is the value used in the largest number of the clinical trials in the sub-database as shown in Table 1. In this case the desirable value corresponding to the minimum risk is equal to the mode value.

Identification of Value for Upper Age Limit: There are 163 trials that include Upper Age Limit as a parameter. As shown in Table 2, “N/A” (no upper age limit) was specified in 142 trials as a value for Upper Age Limit. Thus, the value for Upper Age Limit is determined to be “N/A”.

TABLE 1
Value Identification of Lower Age Limit

| | Frequency of such |
|---|---|
| Value of Lower | value in |
| Age Limit | Clinical Trials |
| 18 years | 148 |
| 20 years | 8 |
| 65 years | 1 |
| 70 years | 5 |
| N/A | 1 |
| Total | 163 |

TABLE 2
Value Identification of Upper Age Limit

| Value of Upper | Frequency of such |
|---|---|
| Age Limit | value in Clinical Trials |
| N/A | 142 |
| 70 years | 6 |
| 75 years | 6 |
| 120 years | 3 |
| 74 years | 3 |
| 99 years | 2 |
| 80 years | 1 |
| Total | 163 |

Value Identification of Disease Stage: A sub-database contains 147 trials that include Disease Stage as a parameter for the inclusion/exclusion criteria as shown in Table 3. Among them, a disease stage of “IIIB/IV” is specified in 78 trials. The value for Disease Stage is determined to be “IIIB/IV”.

TABLE 3
Value Identification of Disease Stage

| Value of | Frequency of such value |
|---|---|
| Disease Stage | in Clinical Trials |
| IIIB/TV | 78 |
| IV | 22 |
| IIIB | 14 |
| III/IV | 7 |
| III | 6 |
| IB-IIB | 3 |
| I-III | 2 |
| II-IV | 2 |
| I-IV | 2 |
| Others | 11 |
| Total | 147 |

Value Identification of ECOG Performance Score: This is a common parameter of inclusion/exclusion criteria in cancer clinical trials as shown in Table 4. In 144 trials that include ECOG Performance Score (also ECOG Score), 81 trials included NSCLC patients with ECOG Performance Score 0 and 1. The value for ECOG Performance Score is determined to be “0 and 1”.

TABLE 4
Value Identification of ECOG Performance Score

| Value of ECOG | Frequency of such value in |
|---|---|
| Performance Score(s) | Clinical Trials |
| 0 and 1 | 81 |
| 0, 1 and 2 | 53 |
| 1 | 3 |
| 2 | 3 |
| 0, 1, 2 and 3 | 2 |
| 1 and 2 | 1 |
| 2 and 3 | 1 |
| Total | 144 |

Value Identification of Life Expectancy: In a sub-database of 58 trials that include Life Expectancy as a parameter, 54 trials included patients with Life Expectancy of 3 months or longer. The value for Life Expectancy is determined to be “3 months or longer”.

Similarly, an exhaustive list of inclusion/exclusion criteria for a Phase II NSCLC clinical trial can be well planned.

In one embodiment, a specific parameter can be added if such addition fits the objectives of the target clinical trial. In one embodiment, a specific parameter can be removed if such removal fits the objectives of the target clinical trial. For example, majority of the 178 trials did not include life expectancy, whether such parameter is necessary for the protocol can be evaluated by a quantitative analysis.

While a set of comprehensive inclusion/exclusion criteria can be pragmatically developed for a clinical trial protocol, there is no “one size fit all” approach that can practically work in all clinical development. In one embodiment, a set of comprehensive inclusion/exclusion criteria can serve well as a starting template. These inclusion/exclusion criteria, however, may need to be further verified and/or modified to fit one or more objectives of a specific clinical trial. These objectives include, but are not limited to, a medical need, a regulatory authority requirement, or a combination of several factors.

The impact of a potential modification on the objectives of a clinical trial can be quantitatively described. One typical quantitative relationship between GSER and N was disclosed by LI in US publication No. 20160042155.

### Example 3

In one embodiment, the inclusion/exclusion criteria are further verified by comparing the patient characteristics that result from using the inclusion/exclusion criteria based on historical data with those of patients at baseline and modifying (or fine tuning) inclusion/exclusion criteria if necessary.

TABLE 5
Status of ECOG Performance Score

| Score | ECOG Performance Status |
|---|---|
| 0 | Fully active, able to carry on all pre-disease performance |
| | without restriction |
| 1 | Restricted in physically strenuous activity but ambulatory |
| | and able to carry out work of a light or sedentary nature, e.g., |
| | light house work, office work |
| 2 | Ambulatory and capable of all selfcare but unable to carry |
| | out any work activities; up and about more than 50% of |
| | waking hours |
| 3 | Capable of only limited selfcare; confined to bed or chair |
| | more than 50% of waking hours |
| 4 | Completely disabled; cannot carry on any selfcare; totally |
| | confined to bed or chair |
| 5 | Dead |

In one embodiment, the information on a group patient meeting the filter parameters (i.e., the primary inclusion/exclusion criteria) is collected into a sub-database. The characteristics of these recruited/selected patients at the beginning of a clinical trial are patient baseline characteristics. These characteristics are governed by the set of inclusion/exclusion criteria in the protocol, as well as by the epidemiology of that particular disease.

The ECOG performance score as shown in Table 5 is used as an example.

In Example 2, 81 of 144 trials included patients with ECOG scores 0 and 1 whi1e52 of 144 trials included patients with ECOG scores 0, 1, and 2. In other words, ECOG scores 0 and 1 is the mode value. ECOG scores 0, 1, and 2 were also frequently used in trial designs. Using ECOG scores 0, 1, and 2 as inclusion/exclusion criteria may lead to a larger target patient population and allow to complete patient recruitment within a shorter period.

The impact of selecting a particular value of ECOG performance score can be quantitatively evaluated.

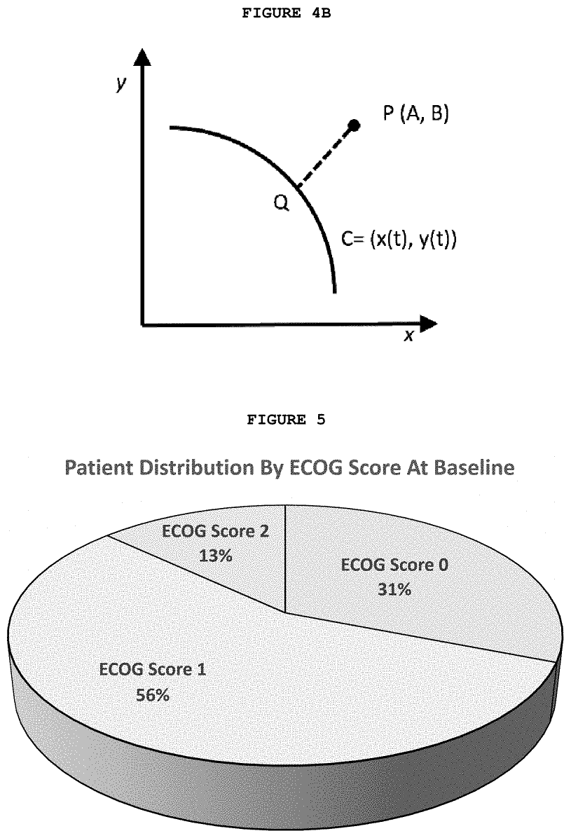

There were 5,415 patients in total as baseline with ECOG scores of 0, 1 or 2 selected from 35 Phase II NSCLC clinical trials. Among them, as shown in FIG. 5, there were 1,654 (30.5%), 3,046 (56.3%), and 715 (13.2%) patients corresponding to ECOG score of 0, 1 and 2, respectively.

In one embodiment, as shown in Table 6, expanding patient population to include those with ECOG score of 2 led to a reduction of median enrollment cycle time by 11.1% from 577 days to 513 days as calculated based on the historical data. The reduction of enrollment cycle time (11.1%) is proportional to the expansion of patient population (13.2%). In one embodiment, the median is the value separating the higher half from the lower half of a data sample.

TABLE 6
Influence of ECOG score adjustment to ECT

| | Median Enrollment Cycle Time (days) |
|---|---|
| ECOG Score 0, 1 | 513 |
| ECOG Score 0, 1, 2 | 577 |
| All | 618 |

In one embodiment, the desirable value is changed in view of the objective(s) and priorities of the clinical trial. If one of the objectives is to achieve a shorter ECT and a larger population, ECOG score 0, 1, 2 should be selected, i.e., the desirable value for ECOG Score in this scenario is not the mode value, but the second most frequently used value. If the objective is to target a narrowly defined population, ECOG score 0 and 1 should be selected, i.e., the desirable value for ECOG Score is the mode value.

In one embodiment, the further modification approach described above is applied to other parameters. In one embodiment, a clinical trial with inclusion/exclusion criteria targeting a larger patient population does not always lead to shorter ECT. The composition of patient population and/or evolving standard of care are some examples of factors that can potentially overpower the size of targeted patient population. When all the other factors have equal or similar effect, an incremental expansion of patient population may lead to reduction of enrollment cycle time.

The inclusion/exclusion criteria to be further adjusted must be extensively studied by the medical community.

### Example 4

### Identify and Avoid Risky Inclusion/Exclusion Criteria

Clinical trials are often required to enroll a narrowly defined small portion of patient population with a disease indication. Such trials may be termed trials in special populations. It is well understood that these clinical trials are operationally difficult to execute. There is currently no quantitative method to identify and measure operational risks. Further, there is no way to communicate these risks among stakeholders of clinical trial sponsors and to regulatory authorities around the world. These obstacles often lead to extremely prolonged enrollment cycle times and/or trial failure. Sometimes such clinical trials fail because the targeted or defined patient population does not exist, or is too small to recruit sufficient number of patients in a reasonable time frame.

In one embodiment, the present invention provides an approach to identify appropriate inclusion/exclusion criteria for such trials by mapping out the relationship among Gross Site Enrollment Rate (GSER), Investigator Sites (N), and the enrollment in each clinical trial in one chart. The approach measures the operational risk(s) related to inclusion/exclusion criteria, and/or risk(s) that may lead to clinical trial failure. In one embodiment, the relationship between GSER and N for a clinical trial meeting all criteria in Example 1 can be described as:

GSER=2.5394*N^{-0.738}.

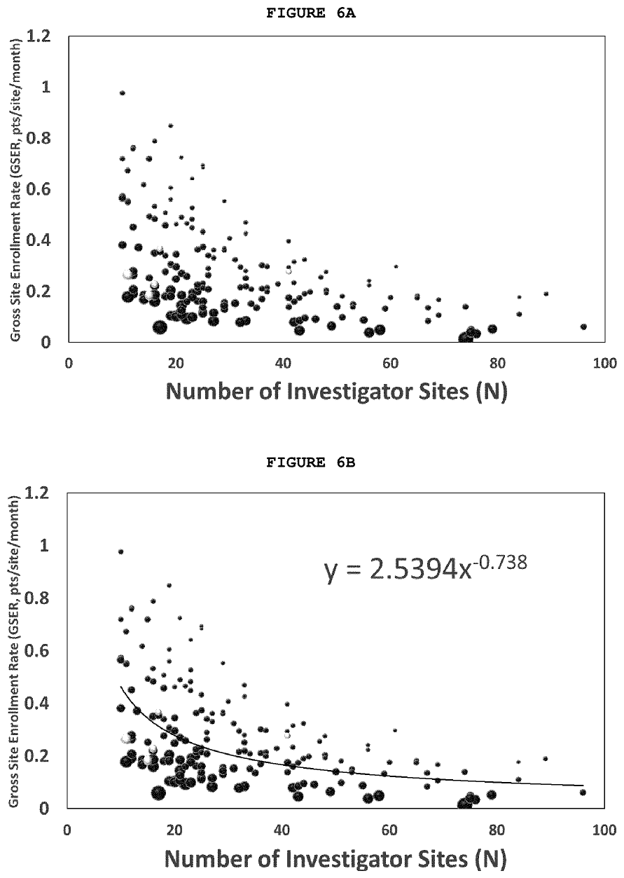

Age: In Example 2, the starting desirable values for lower age limit and upper age limit are “18” and “N/A”, respectively. If the objective of the target clinical trial is to focus on a narrowly defined group, it will introduce various risks with various degrees of impact on the operational feasibility. For example, there were 5 of 163 Phase II NSCLC trials targeting senior patients with an age of 70 years or older. These trials correspond to light-colored bubbles in FIG. 6A. According to the GSER bubble chart as shown in FIG. 6A, three (3) of the above mentioned five (5) clinical trials were really off the pattern, i.e., the corresponding GSER was way below the ideal curve, which may require a much longer ECT to complete the targeted total enrollment.

The present invention provides a new quantitative method describing the pattern in FIG. 6B. The quantitative relationship can help users visually to easier understand the risk associated with selecting particular inclusion/exclusion criteria and to quantify the risk. FIG. 6B depicts the risk introduced by restricting ages of eligible patients.

The median enrollment cycle time for the trials including senior patients 70 years and older is 822 days, while the median enrollment cycle time for the entire 178 trials is 618 days. Thus, selecting 70 years as the value for the upper age for a target clinical trial is associated with the risk of impacting the ECT. If a shorter ECT is one of the objectives of the target clinical trial, then selecting 70 years for the upper age limit is associated with a higher risk of not achieving the objective of the target clinical trial.

### Example 5

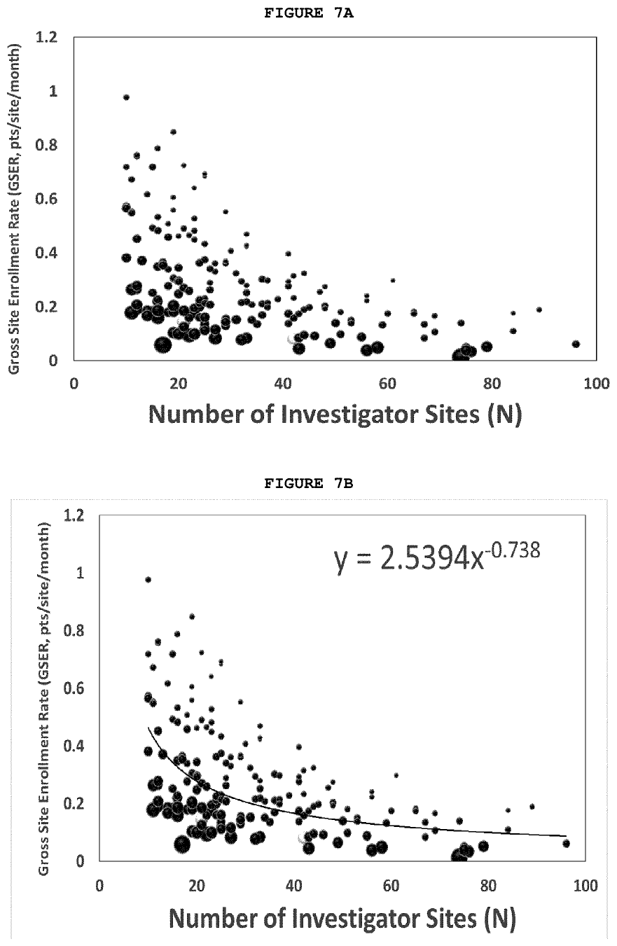

ECOG score: In Example 2, the starting inclusion/exclusion criteria include “0 and 1” as the value for ECOG score. Using the verification/modification method based on baseline patient characteristics as described in Example 3, expanding ECOG score to 0, 1, and 2 leads to a shorter ECT. By contrast, clinical trials that used ECOG score of 2 as inclusion/exclusion criteria had a dramatically longer ETC. Two (2) of the Phase II NSCLC trials targeted to enroll patients with ECOG score of 2 are shown in light-colored bubbles are significantly off the pattern according to FIG. 7A.

In one embodiment, a quantitative relationship objectively describing the risk is shown in FIG. 7B. The quantitative relationship can help users visually to easier understand the risk associated with selecting particular inclusion/exclusion criteria and to quantify the risk. FIG. 7B depicts the risk introduced by restricting ECOG score of eligible patients. The median enrollment cycle time for the trials including only patients with ECOG score 2 is 1,445 days, while the median enrollment cycle time for the entire 178 trials is 618 days.

In one embodiment, for a Phase 2 NSCLC clinical trial to target patient population to those older than 70 years old, or target patient population to those with ECOG performance score of 2 (we call them special population clinical trial) means introducing quantifiable risk, resulting in a significantly longer ECT.

### Example 6

Interplay among factors: In one embodiment, the value of certain parameter(s) may significantly affect the values of the others. There is currently no way to describe and/or quantify the risks. In the database, there is one (1) Phase II NSCLC clinical trial targeted to include patients 70 years and older and with ECOG score of 2. With an initial plan to enroll 121 patients, the clinical trial was terminated after enrolling 54 patients with a note that “Study was stopped due to slower than expected recruitment.” By using the method described here, the potential risks could have been detected, which may potentially have saved $15 million.

In one embodiment, the method described above can be expanded to designing a clinical trial protocol for any clinical trial or optimizing the design of an existing protocol. In the above example, a set of inclusion/exclusion criteria for a pancreatic cancer trial protocol was examined. As can be seen in Table 7, some parameter values deviated from previous clinical trial, which prompted objective discussion from the team and resulted in an improved design.

TABLE 7
A set of inclusion/exclusion criteria for a pancreatic cancer
trial according to one embodiment of the present invention.

| | | | | Total |
|---|---|---|---|---|
| Parameter | Original value | Desirable value | Frequency | frequency |
| Disease | Histological or | histologically or | 21 | 27 |
| characteristics | cytologically | cytologically | | |
| 1 | confirmed | confirmed | | |
| Disease | Unresectable | Unresectable | 17 | 27 |
| characteristics | | | | |
| 2 | | | | |
| Age | 18 and older | 18 and older | 20 | 27 |
| Performance | Karnofsky | ECOG | 18 | 26 |
| score | | | | |
| ECOG score | 0 1 2 | 0 1 2 | 17 | 26 |
| Life expectancy | NA | 3 months | 7 | 12 |
| Adequate organ | Hemoglobin | Hemoglobin | 5 | 7 |
| function 1 | ≥9.0 g/dL | ≥9.0 g/dL | | |
| Adequate | absolute | absolute | 8 | 10 |
| organ | neutrophil count | neutrophil count | | |
| function 2 | 1,500/mm3 | 1,500/mm3 | | |
| Adequate organ | Platelet count | Platelet count | 12 | 13 |
| function 3 | ≥100,000/mm3 | ≥100,000/mm3 | | |
| Adequate organ | 1× ULN | Bilirubin ≤1.5× | 5 | 10 |
| function 4 | | ULN | | |
| Adequate organ | 1.5× ULN | Creatinine | 5 | 10 |
| function 5 | | ≤1.5× ULN | | |
| Adequate organ | NA | White blood | 3 | 6 |
| function 6 | | cell count | | |
| | | ≥3500/mm3 | | |

Note:
the frequency is the number of times a value has been used in clinical trials containing such parameter in the sub-database; total frequency is the total number of clinical trials containing such parameter in the sub-database.

In one embodiment, as shown in Table 7, a set of inclusion/exclusion criteria is established based on a quantitative analysis of the present invention. In comparison to the original values in a previous protocol listed in the second column of Table 7, the present invention selects ECOG as value for performance score rather than Karnofsky. Life expectancy is a newly selected parameter, a value for life expectancy is set as “3-months”; the desirable value for Bilirubin level is “1.5×ULN” rather than “1×ULN”; “white blood cell count” is also another newly selected parameter with the desirable value of “3500/mm3”.

1-22. (canceled)

23. A system for developing a set of inclusion/exclusion criteria for a target clinical trial related to a disease or a condition, said system comprising:
a storage unit;
a computing unit;
an output unit; and
an input unit, all operable together,
wherein a filter comprising a set of filtering parameters is provided through said input unit;
wherein said filter is applied to a master database comprising historical data on clinical trials to create a sub-database in said storage unit, said sub-database comprising historical data on clinical trials related to said disease or said condition and having sufficient data for subsequent analysis,
wherein said computing unit conducts the steps of:
a) selecting parameters in said sub-database to obtain a number of selected parameters, and
b) conducting an analysis on said selected parameters to determine their desirable values, wherein said analysis comprises:
1) a frequency analysis to determine the frequency with which a value associated with a selected parameter has been used in the clinical trials in said sub-database, and
2) a quantitative analysis to quantify a risk associated with selecting a value associated with a selected parameter, or a risk associated with selecting multiple values, wherein each of said multiple values is associated with one of the selected parameters; wherein a value associated with a selected parameter and an acceptable risk is a desirable value, and wherein one or more selected parameters with their desirable values define a set of inclusion/exclusion criteria; and
wherein said output unit transmits and displays said set of inclusion/exclusion criteria.

24. The system of claim 23, wherein said filter comprises at least one parameter that can be used to characterize said target clinical trial.

25. The system of claim 24, wherein said at least one parameter is selected from the group consisting of type/stage of disease/disorder, age, gender, phase of the clinical trial, country, number of clinical trials, number of patients, number of investigator sites, enrollment cycle time (ECT), Site Effectiveness Index (SEI), Adjusted Site Enrollment Rate (ASER), and Gross Site Enrollment Rate (GSER).

26. The system of claim 23, wherein said quantitative analysis analyzes how different values of a selected parameter in said sub-database affect changes in one or more characters, each of which is an operational outcome of clinical trials in said sub-database, wherein said analysis may be repeated for other selected parameters.

27. The system of claim 26, wherein said operational outcome is selected from the group consisting of number of patients, number of investigator sites, ECT, SEI, ASER, and GSER.

28. The system of claim 23, wherein said quantitative analysis evaluates the risk associated with selecting a value for a selected parameter or the risk associated with selecting multiple values for multiple selected parameters in view of the distance between a curve of an equation and a point corresponding to said value for said selected parameter or said multiple values for said multiple selected parameters, wherein said equation quantitatively describes a relationship between two variables, wherein at least one of said variables is or is associated with a character.

29. The system of claim 28, wherein a value for said selected parameter or multiple values for said multiple selected parameters are chosen as a desirable value or desirable values if they are aligned with one or more objectives of said target clinical trial, resulting in a set of inclusion/exclusion criteria for said target clinical trial.

30. The system of claim 29, wherein said one or more objectives are selected from the group consisting of number of investigator sites, number of patients, ECT, SEI, ASER, GSER, and targeted patient population.

31. The system of claim 23, wherein at least one of said one or more desirable values is most frequently used in the clinical trials in said sub-database.

32. The system of claim 23, wherein one or more of the selected parameters in step a) have been used in at least 50% of the clinical trials in said sub-database.

33. A method of developing a set of inclusion/exclusion criteria for a clinical trial related to a disease or a condition, said method comprising:
a) applying a filter to a master database containing historical data on clinical trials to create a sub-database, wherein said filter comprises a set of filtering parameters, and said sub-database contains clinical trials related to said disease or condition, and has sufficient data for subsequent analysis,
b) selecting parameters that fit the objectives of the target clinical trial from said clinical trials in said sub-database to obtain a number of selected parameters;
c) conducting an analysis to determine desirable values of said selected parameters, wherein said analysis comprises:
1) a frequency analysis to identify the frequency with which a value associated with a selected parameter has been used in the clinical trials in said sub-database, and
2) a quantitative analysis to quantify a risk associated with selecting a value for such selected parameter, or a risk associated with selecting values for such selected parameters, wherein each of said multiple values is associated with one of the selected parameters; wherein a value associated with a selected parameter and an acceptable risk is a desirable value, and wherein multiple selected parameters with their desirable values define a set of inclusion/exclusion criteria, and
d) outputting said set of inclusion/exclusion criteria.

34. The method of claim 33, wherein said filter comprises at least one parameter that can be used to characterize said clinical trials or said target clinical trial.

35. The method of claim 34, wherein said at least one parameter is selected from the group consisting of type/stage of disease/disorder, age, gender, phase of the clinical trial, country, number of clinical trials, number of patients, number of investigator sites, ECT, SEI, ASER, and GSER.

36. The method of claim 33, wherein said quantitative analysis analyzes how different values of a selected parameter in said sub-database affect changes in one or more characters, each of which is an operational outcome of clinical trials in said sub-database, wherein said analysis may be repeated for other selected parameters.

37. The method of claim 36, wherein said operational outcome is selected from the group consisting of number of patients, number of investigator sites, ECT, SEI, and ASER, and GSER.

38. The method of claim 33, wherein said quantitative analysis evaluates the risk associated with selecting a value for a selected parameter or the risk associated with selecting multiple values for multiple selected parameters in view of the distance between a curve of an equation and a point corresponding to said value for said selected parameter or said multiple values for said multiple selected parameters, wherein said equation quantitatively describes a relationship between two variables, wherein at least one of said variables is or is associated with a character.

39. The method of claim 38, wherein a value for said selected parameter or multiple values for said multiple selected parameters are chosen as a desirable value or desirable values if they are aligned with one or more objectives of said target clinical trial, resulting in a set of inclusion/exclusion criteria for said target clinical trial.

40. The system of claim 39, wherein said one or more objectives are selected from the group consisting of number of investigator sites, number of patients, ECT, SEI, ASER, GSER, and targeted patient population

41. The method of claim 33, wherein one or more of said desirable values are most frequently used in the clinical trials in said sub-database.

42. The method of claim 33, wherein one or more of said selected parameters in step b) have been used in at least 50% of the clinical trials in said sub-database.
